(19) United States
(12) Patent Application Publication
(10) Pub. No.: US 2024/0299724 A1
(43) Pub. Date: Sep. 12, 2024

(54) RESPIRATORY SWIVEL CONNECTOR

(71) Applicant: Nutic LLC, Kansas City, KS (US)

(72) Inventor: Matthew Young, Charlotte, NC (US)

(21) Appl. No.: 18/599,882

(22) Filed: Mar. 8, 2024

Related U.S. Application Data
(60) Provisional application No. 63/489,460, filed on Mar. 10, 2023.
(60) Provisional application No. 63/581,163, filed on Sep. 7, 2023.

(51) Int. Cl. A61M 39/10; A61M 39/14

(52) U.S. Cl. CPC A61M 39/1055; A61M 39/14

(57) ABSTRACT

Swivel connection devices are provided that are configured for attachment to a range of respiratory equipment, including respiratory tubes and fittings, particularly those associated with a nebulizer for delivering medication to a patient. The swivel connection devices are universal in that they can accommodate connection to multiple sizes of respiratory equipment.

### RELATED APPLICATIONS

This application claims the priority benefit of U.S. Provisional Patent Application No. 63/489,460, filed Mar. 10, 2023, and U.S. Provisional Patent Application No. 63/581,163, filed Sep. 7, 2023, both of which are incorporated by reference herein in their entireties.

### BACKGROUND OF THE INVENTION

### Field of the Invention

The present invention relates to respiratory tube fittings and more particularly to a swivel device which can be installed between a range of respiratory tubes or fittings. The device can be for applications where a T-Valve is used in conjunction with a nebulizer to deliver medication to a patient through the lungs. Often times, the tubing memory causes the T-Valve to rotate in conjunction with the hose causing the medication to spill out of the nebulizer and as a result, the patient does not receive the full dose of medication.

### Description of the Prior Art

In recent years, the number of respiratory health concerns have continued to increase. In order to treat these respiratory conditions, often times medication is provided through the airway and directly into the patient's lungs. This is often the case for mechanical ventilated, tracheostomy, laryngectomy, and CPAP/Bi-Level patients through the use of a nebulizer connected to the respiratory tubing which helps patents to breath.

Conventional respiratory fittings to connect T-Valves and nebulizers to the hoses used to transmit the medications are rigid and as a result transfer the twisting of the hose into the nebulizer causing the medical to spill or leak out of the device. Moreover, conventional fittings are configured for use with a single size of hose connections requiring hospitals and medical offices to stock multiple types of connector fittings. Thus, there is a need for a swiveling connector for a respiratory therapy apparatus which is able to accommodate multiple sizes of hose fittings, is not easily disconnected from the ventilation tube and/or the nebulizer, and which forms a gas-tight seal in the swivel area.

### SUMMARY OF THE INVENTION

The present invention describes a novel device which enables a range of respiratory hoses with differently sized connection fittings to be connected to T-fittings/valves and/or drug delivery systems and ensure that the proper orientation is maintained such that the prescribed amount of medication is distributed through the respiratory treatment system.

More specifically, the device is designed in such a manner that allows either end of the device to accept multiple size male or female fittings. The first open end is configured to have at least two different wall thicknesses which form the connection points for the male side of the swivel device. The second end of the device is comprised of a step feature with a dual inside diameter connection point to again accommodate multiple sized hose fittings or valves. These two ends are coupled together with a latch type connection which ensures positive engagement of the two components. A tight friction fit between the two components over a given distance ensures a low-pressure air-tight seal without the need for a secondary seal material.

According to one embodiment of the invention there is provided a connector comprising a first section having first and second male coupling surfaces. The first male coupling surface has a smaller outer diameter than the second male coupling surface. The swivel connector further comprises a second section having first and second female coupling surfaces. The first female coupling surface has a smaller inner diameter than the second female coupling surface. The first and second sections are connected by a swivel interface to permit independent rotation of the first and second sections relative to each other.

According to another embodiment of the invention there is provided a connector comprising a first section comprising first and second male coupling ends, and a second section comprising first and second female coupling ends. The first male coupling end has at least first and second male coupling surfaces, with the first male coupling surface being located outboard from the second male coupling surface and having an outer diameter that is smaller than the second male coupling surface. The first female coupling end has at least first and second female coupling surfaces, with the first female coupling surface being located outboard from the second female coupling surface and having an inner diameter that is larger than the second female coupling surface. The second male coupling end and the second female coupling ends form a swivel connection when mated together permitting independent rotation of the first and second sections relative to each other.

According to another embodiment of the present invention there is provided a method of joining two pieces of respiratory equipment together. A connector is provided comprising a first section and a second section. The first section comprises first and second male coupling surfaces, the first male coupling surface having a smaller outer diameter than the second male coupling surface. The second section comprises first and second female coupling surfaces, the first female coupling surface having a smaller inner diameter than the second female coupling surface. The first and second sections are connected by a swivel interface to permit independent rotation of the first and second sections relative to each other. A female end of a first piece of respiratory equipment is attached to the first section, and a male end of a second piece of respiratory equipment to the second section.

According to still a further embodiment of the present invention there is provided a connector comprising a first section having first and second coupling ends, and a second section having an outer sealing surface. The first coupling end comprises a female coupling end with an inner sealing surface, and the second coupling end comprises first and second male coupling surfaces. The first male coupling surface has a smaller outer diameter than the second male coupling surface. The first and second sections are connected by a swivel interface to permit independent rotation of the first section relative to the second section. The swivel interface including the inner sealing surface of the first section and the outer sealing surface of the second section.

According to yet another embodiment, an assembly is provided that comprises a connector according to any embodiment described herein, a first piece of respirator equipment attached to the first section, and a second piece of respiratory equipment attached to the second section.

### BRIEF DESCRIPTION OF THE DRAWINGS

FIG. 1 is a cross-sectioned view of a connector according to one embodiment of the present invention;

FIG. 2 is an exploded view of the connector of FIG. 1;

FIG. 3 is a perspective view of the assembled connector of FIG. 1;

FIG. 4 is an alternate perspective view of the assembled connector of FIG. 1;

FIG. 5 is an exploded view of a tee and hose assembly including the connector of FIG. 1;

FIG. 6 is a cross-sectioned view of an alternate connector embodiment having a reduced length;

FIG. 7 is an exploded view of the connector of FIG. 6;

FIG. 8 is a perspective view of the connector of FIG. 6;

FIG. 9 is an exploded view of two of the connectors of FIG. 6 in use with a conventional tee;

FIG. 10 is an exploded view of a tee and hose assembly including the connectors of FIG. 6;

FIG. 11 is an exploded view of a swivel tee connector in accordance with an embodiment of the present invention;

FIG. 12 is a perspective view of the tee connector of FIG. 11;

FIG. 13 is an exploded view of a tee and hose assembly including the connector of FIG. 11; and

FIG. 14 is a cross-sectioned view of the tee connector of FIG. 11.

### DETAILED DESCRIPTION OF THE PREFERRED EMBODIMENT

The drawing figures do not limit the current invention to the specific embodiments disclosed and described herein. While the drawings do not necessarily provide exact dimensions or tolerances for the illustrated components or structures, the drawings are to scale as examples of certain embodiments with respect to the relationships between the components of the structures illustrated in the drawings.

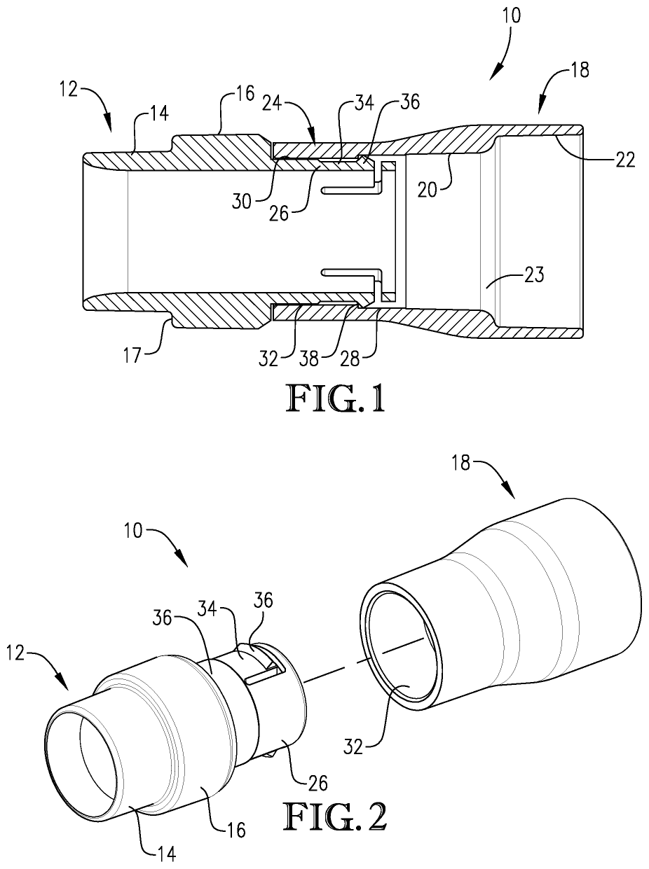

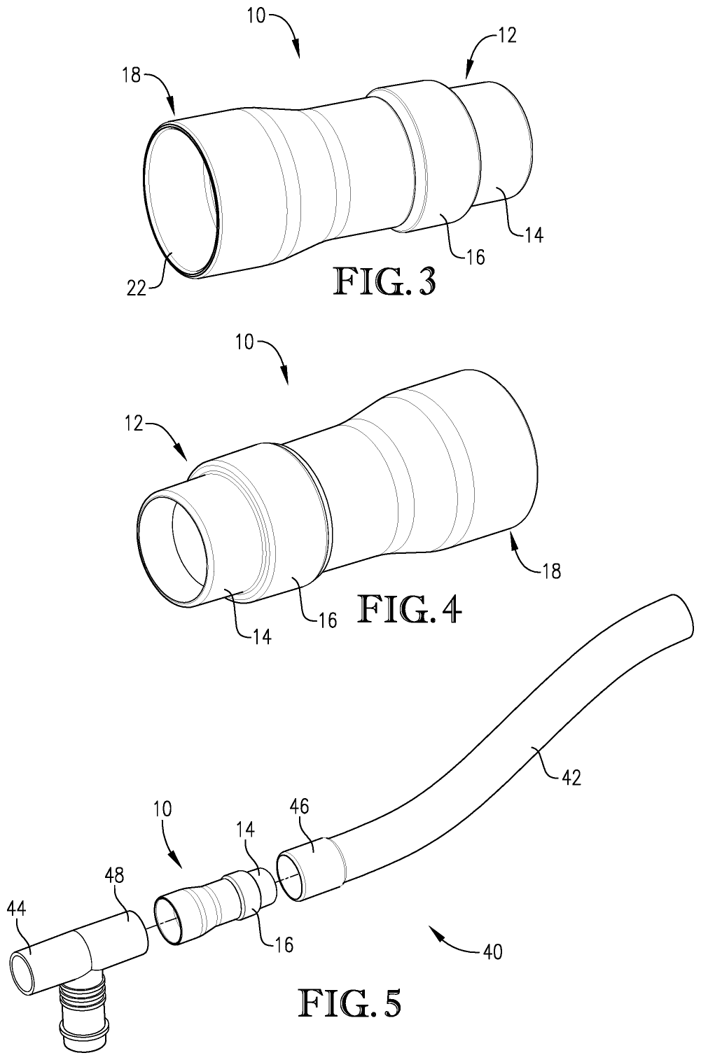

FIGS. 1-4 illustrate a swivel connector 10, preferably a straight connector, according to one embodiment of the present invention. The connector comprises a first section 12 having a first male coupling surface 14 and a second male coupling surface 16. The first male coupling surface 14 comprises a smaller outer diameter (e.g., 18 mm) than the second male coupling surface 14 (e.g., 22 mm) thereby permitting the first section 12 to accommodate attachment to female hose ends or valve fittings, for example, of different internal diameters. In one or more embodiments, the first male coupling surface 14 is located outboard of the second male coupling surface 16. The first and second male coupling surfaces 14, 16 are connected by an outwardly projecting shoulder 17. In one or more embodiments, each of the first and second male coupling surfaces 14, 16 are of constant outer diameter across the widths thereof, and preferably across the entirety of the widths thereof.

Connector 10 also comprises a second section 18 having a first female coupling surface 20 and a second female coupling surface 22. The first female coupling surface 20 has a smaller inner diameter than the second female coupling surface 22 thereby permitting the second section 18 to be connected with male hose ends or valve fittings, for example, having different outer diameters. In one or more embodiments, the first female coupling surface 20 is located inboard of the second female coupling surface 22. The first and second female coupling surfaces 20, 22 are connected by a radially expanding shoulder 23. In one or more embodiments, each of the first and second female coupling surfaces 20, 22 are of constant inner diameter across the widths thereof, and preferably across the entirety of the widths thereof.

The first section 12 and the second section 18 are connected by a swivel interface 24 that permits independent rotation of the first section 12 and the second section 18 relative to each other. Specifically, the swivel interface comprises a male coupling end 26 that is configured to mate with a female coupling end 28. The male coupling end 26 includes an outer sealing surface 30 that is configured to frictionally engage an inner sealing surface 32 of the female coupling end 28. In one or more embodiments, the outer sealing surface has a width that is at least 15%, at least 20%, or at least 25% of the inner diameter of the first section 12. In one or more embodiments, the inner sealing surface 32 has a width that is greater than the width of the outer sealing surface. Sealing surfaces 30, 32 form a low-pressure, airtight seal that is operable to inhibit or prevent the escape of gases, such as respiratory gases or medications, flowing through connector 10 through the swivel interface 24.

In order to ensure first section 12 and second section 18 remain fastened together, the male coupling end 26 can include at least one tab 34 including an outer lip 36 that is configured to engage with a shoulder 38 that is formed in the female coupling end 28 when the first section 12 and the second section 18 are connected. In one or more embodiments, the swivel interface 24 is configured such that a stream of gas passing through the connector 10 at a pressure of between 10 to 15 psia experiences a pressure drop due to loss of the stream of gas through the swivel interface 24 of less than 1 psia, less than 0.5 psia, or less than 0.25 psia.

In one or more embodiments, at least one, and preferably all, of the first and second male coupling surfaces 14, 16, first and second female coupling surfaces 20, 22, and swivel interface 24 do not comprise pipe threads or barbs. Accordingly, in one or more embodiments, at least one, and preferably all, of the first and second male coupling surfaces 14, 16, first and second female coupling surfaces 20, 22, and swivel interface 24 comprise friction mating surfaces.

Connector 10 can be used to attach various respiratory equipment components together. In particular, the respiratory equipment components may comprise hoses, tubing, and other fittings, such as tee fittings and tee valves. In preferred embodiments, an assembly of components can be made that includes connector 10 disposed in between first and second pieces of respirator equipment. This assembly can also include, or be configured for attachment to, a nebulizer for delivery of a medication to a patient. FIG. 5 depicts an exemplary assembly 40 comprising connector 10, a hose 42, and a tee fitting 44. Because of the multiple diameters of the various male and female coupling surfaces provided, connector 10, as a standalone device, can accommodate interconnection between a variety of hose and fitting sizes.

In one embodiment, a connector 10 is provided. A female end of a first piece of respiratory equipment, e.g., female end 46 of hose 42, is attached to one of the first and second male coupling surfaces 14, 16 of first section 12. A male end of a second piece of respiratory equipment, e.g., male end 48 of tee fitting 44, is attached to one of the first and second female coupling surfaces 20, 22 of second section 18. Thus, the connector 10 permits tee fitting 44 and hose 42 to independently swivel without changing the orientation of the other.

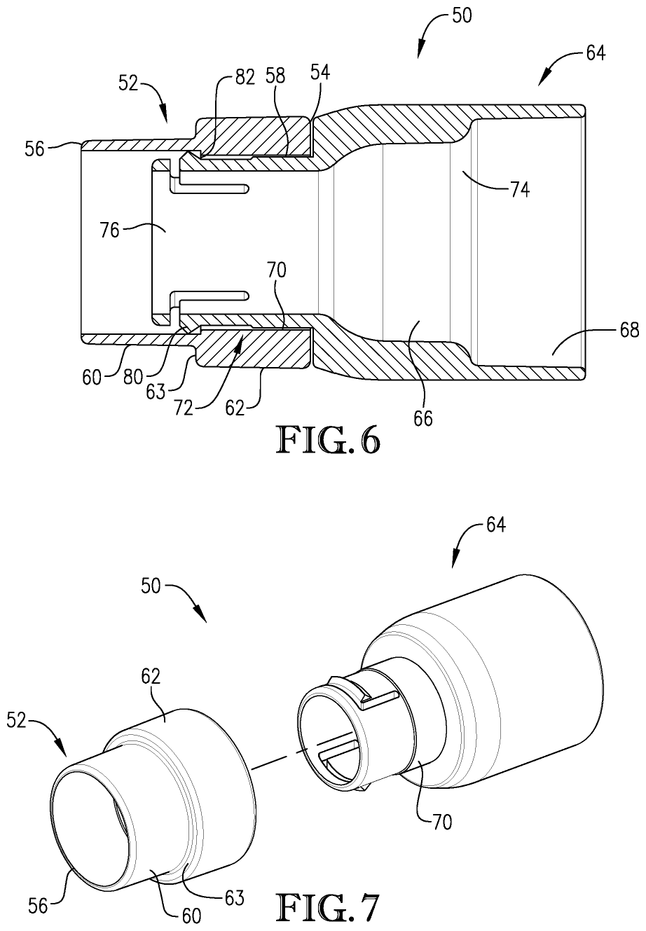

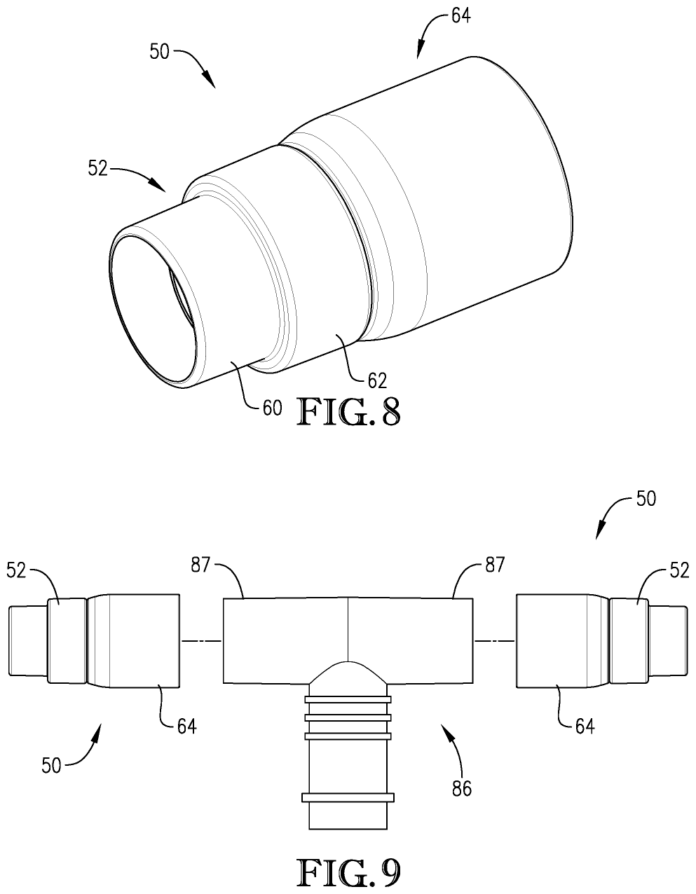

Another embodiment of a connector in accordance with the present invention is illustrated in FIGS. 6-8. Connector 50, preferably a straight connector, comprises a swivel fitting that may be shorter in overall length relative to connector 10, which may further assist in reducing hose binding which occurs as a result of hose memory in the respiratory circuit. In connector 50 the overall length of the connector is reduced by placing the male connector portion onto the female hose connector portion and embedding the female portion of the connector within the male hose connection portion. This aspect enables length reduction by having both the male hose connection and female swivel fitting occupying the longitudinal space within the connector. For example, in one or more embodiments, such changes (e.g., embedding the female swivel connection in the male hose connection) allows the overall length of the fitting to be reduced by 20-30% or more.

Connector 50 comprises a first section 52 having a first coupling end 54 and a second coupling end 56. The first coupling end 54 comprising a female coupling end with an inner sealing surface 58. The second coupling end 56 comprises a first male coupling surface 60 and a second male coupling surface 62 that are connected by an outwardly projecting shoulder 63. The first male coupling surface 60 has a smaller outer diameter than the second male coupling surface 62. Further, the first male coupling surface 60 is located outboard of the second male coupling surface 62. In one or more embodiments, each of the first and second male coupling surfaces 60, 62 are of constant outer diameter across the widths thereof, and preferably across the entirety of the widths thereof.

Connector 50 comprises a second section 64 having an outer sealing surface 70 that is configured to engage inner sealing surface 58 of first section 52. In the embodiment of FIG. 6, the second section 64 has a first female coupling surface 66 and a second female coupling surface 68. The first female coupling surface 66 has a smaller inner diameter than the second female coupling surface 68. The first female coupling surface 66 is located inboard of the second female coupling surface 68. Cooperatively outer sealing surface 70 and inner sealing surface 58 frictionally engage to form a swivel interface 72 that permits independent rotation of the first section 52 relative to the second section 64. In one or more embodiments, the first and second female coupling surfaces 66, 68 are connected by a radially expanding shoulder 74. In one or more embodiments, each of the first and second female coupling surfaces 66, 68 are of constant inner diameter across the widths thereof, and preferably across the entirety of the widths thereof.

In order to ensure first section 52 and second section 64 remain fastened together, a male coupling end 76 of second section 64 can include at least one tab 78 including an outer lip 80 that is configured to engage with a shoulder 82 that is formed in first coupling end 54 when the first section 52 and the second section 64 are connected. In one or more embodiments, the swivel interface 72 is configured such that a stream of gas passing through the connector 50 at a pressure of between 10 to 15 psia experiences a pressure drop due to loss of the stream of gas through the swivel interface 24 of less than 1 psia, less than 0.5 psia, or less than 0.25 psia.

In one or more embodiments, the outer sealing surface 70 has a width that is at least 15%, at least 20%, or at least 25% of the inner diameter of the second section 64. In one or more embodiments, the inner sealing surface 58 has a width that is greater than the width of the outer sealing surface 70. Sealing surfaces 58, 70 form a low-pressure, airtight seal that is operable to inhibit or prevent the escape of gases, such as respiratory gases or medications, flowing through connector 50 through the swivel interface 72.

In one or more embodiments, at least one, and preferably all, of the first and second male coupling surfaces 60, 62, first and second female coupling surfaces 66, 68, and swivel interface 72 do not comprise pipe threads or barbs. Accordingly, in one or more embodiments, at least one, and preferably all, of the first and second male coupling surfaces 60, 62, first and second female coupling surfaces 66, 68, and swivel interface 72 comprise friction mating surfaces.

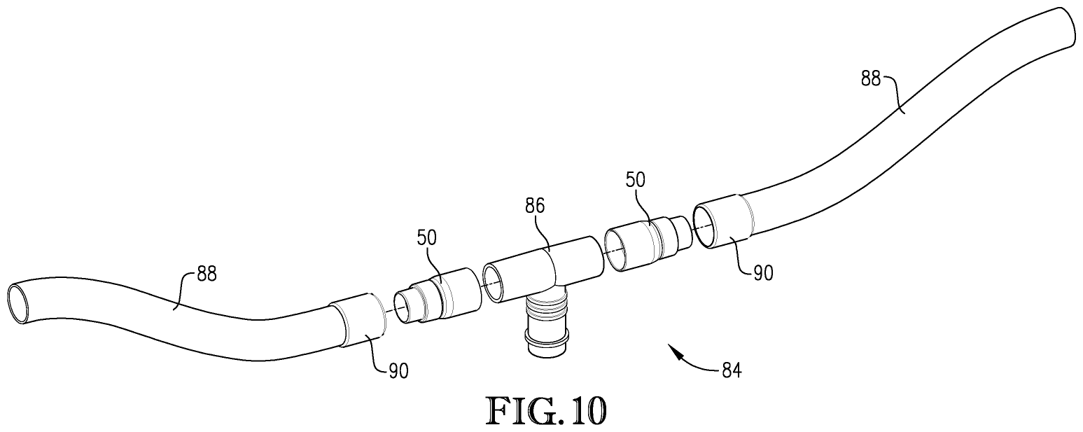

Much like connector 10 described above, connector 50 can be used to attach various respiratory equipment components together. In particular, the respiratory equipment components may comprise hoses, tubing, other fittings, such as tee fittings, and valves. In preferred embodiments, an assembly of components can be made that includes connector 50 disposed in between first and second pieces of respirator equipment. This assembly can also include, or be configured for attachment to, a nebulizer for delivery of a medication to a patient. FIG. 9 depicts an exemplary assembly 84 comprising a pair of connectors 50 and a conventional tee fitting 86, such as a tee valve. Tee fitting 86 comprises horizontally-extending male connectors 87 that are received into a complementary female coupling surface, surface 66 or 68, of second section 64. FIG. 10 further depicts assembly 84 with hoses 88 attached thereto. Hoses 88 are equipped with a female hose end 90 that is configured to engage with a complementary male coupling surface, surface 60 or 62, of first section 52. Because of the multiple diameters of the various male and female coupling surfaces provided, connector 50, as a standalone device, can accommodate interconnection between a variety of hose and fitting sizes.

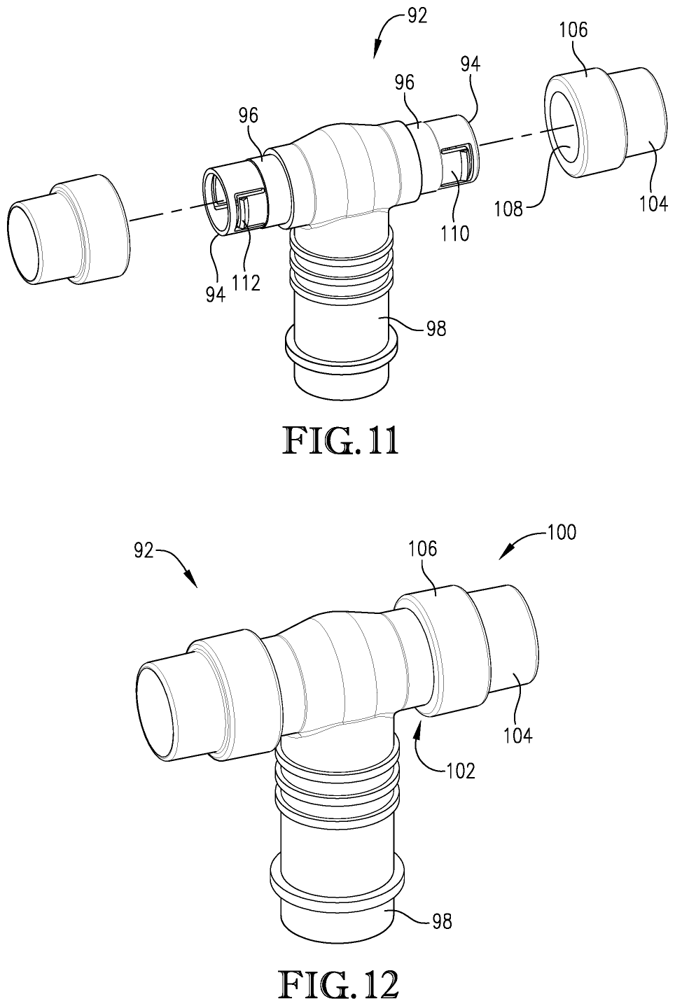

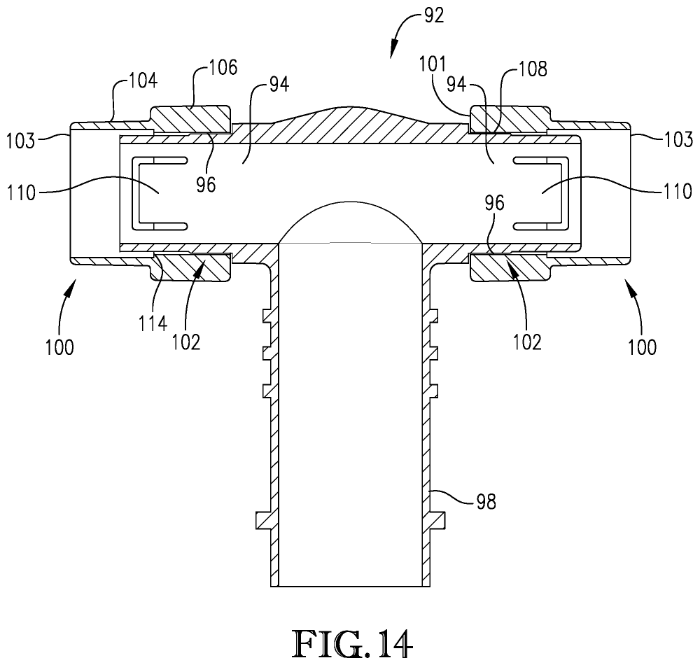

FIGS. 11-14 illustrate a further embodiment in which the connection surfaces of connector 50 have been directly integrated into a tee fitting, thereby eliminating the need for separate connectors and further reducing the overall connection length. The various relationships Turning initially to FIGS. 11 and 14, a tee fitting 92 is provided having spaced part male connection ends 94, each comprising outer sealing surfaces 96. In certain embodiments, the connection ends 94 are linearly spaced apart from each other (along the same axis); however, the connection ends 94 can also extend perpendicularly from the tee fitting 92, if desired. Tec fitting 92 can also comprise a standard-sized connection end 98 (e.g., a non-swivel connection) that is capable of accommodating attachment to various widely available nebulizer systems.

Connector sections 100 can then be attached to male connection ends 94 much in

the same way that first section 52 is attached to second section 64 to form a swivel interface 102. The connector sections 100 comprise a female coupling end 101 provided with an inner sealing surface 108 that is configured to frictionally engage outer sealing surface 96 to provide a low-pressure, airtight seal that is operable to inhibit or prevent the escape of gases, such as respiratory gases or medications, flowing through tee fitting 92 through the swivel interface 102. Connection sections 100 further comprise a male coupling end 103 having a first male coupling surface 104 and a second male coupling surface 106. The first male coupling surface 104 has a smaller outer diameter than the second coupling surface 106. The various dimensions and relationships of the male coupling surfaces and sealing surfaces are similar to those described above for connectors 10 and 50.

To ensure that the tee fitting 92 and connector sections 100 remain fastened together, the male connection ends 94 can include at least one tab 110 including an outer lip 112 that is configured to engage with a shoulder 114 that is formed in the female coupling end 28 when the first section 12 and the second section 18 are connected. In one or more embodiments, the swivel interface 24 is configured such that a stream of gas passing through the connector 10 at a pressure of between 10 to 15 psia experiences a pressure drop due to loss of the stream of gas through the swivel interface 24 of less than 1 psia, less than 0.5 psia, or less than 0.25 psia.

In one or more embodiments, the swivel interface 24 is configured such that a stream of gas passing through the connector 10 at a pressure of between 10 to 15 psia experiences a pressure drop due to loss of the stream of gas through the swivel interface 24 of less than 1 psia, less than 0.5 psia, or less than 0.25 psia.

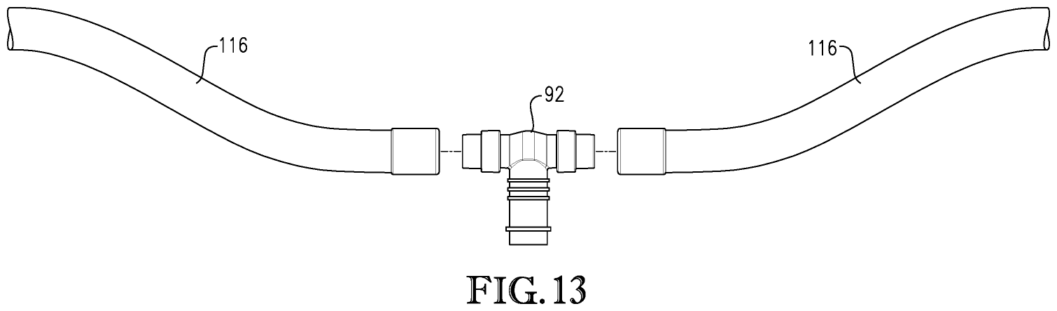

FIG. 13 depicts attachment of hoses 116 to tee fitting 92. The attachment is made in a similar manner as described above in connection with connectors 10 and 50. Advantageously, tee fitting 92 eliminates the need for additional couples to meet the goals of reducing spillage of medication from a nebulizer while being used by a patient but keeping the connection assembly as compact as possible.

As an alternative to the embodiment of tee fitting 92 depicted in FIG. 14, instead of being provided with first and second male coupling surfaces, the tee fitting can be configured with swivel connections having first and second female coupling surfaces to permit connection of the tee fitting with respiratory equipment having male connection ends (as opposed to the female connection ends depicted in FIG. 13). In such an embodiment, the tee fitting would comprise two female sealing surfaces on the horizontal ends thereof, and the connector section would comprise a male sealing surface configured to engage the female scaling surfaces. However, this alternate configuration could result in an increased tee fitting length and weight as compared to tee fitting 92. In still an alternative embodiment, a tee fitting, such as tee fitting 92. could be provided with one male coupling surface and one female coupling surface.

I claim:

1. A connector comprising:
a first section comprising first and second coupling ends, the first coupling end comprising a female coupling end with an inner sealing surface and the second coupling end comprising first and second male coupling surfaces, the first male coupling surface having a smaller outer diameter than the second male coupling surface; and
a second section comprising an outer sealing surface,
wherein the first and second sections are connected by a swivel interface to permit independent rotation of the first section relative to the second section, the swivel interface including the inner sealing surface of the first section and the outer sealing surface of the second section.

2. The connector of claim 1, wherein the second section comprises first and second female coupling surfaces.

3. The connector of claim 2, wherein the first female coupling surface is located inboard of the second female coupling surface.

4. The connector of claim 2, wherein the first and second female coupling surfaces are connected by a radially expanding shoulder.

5. The connector of claim 2, wherein each of the first and second female coupling surfaces are of constant inner diameter across the widths thereof, and preferably across the entirety of the widths thereof.

6. The connector of claim 1, wherein the second section is integral with a tee fitting.

7. The connector of claim 6, further comprising
a third section comprising third and fourth coupling ends, the third coupling end comprising a second female coupling end with a second inner sealing surface and the fourth coupling end comprising third and fourth male coupling surfaces, the third male coupling surface having a smaller outer diameter than the fourth male coupling surface,
a fourth section integral with the tee fitting and comprising a second outer sealing surface, wherein
the third and fourth sections are connected by a second swivel interface to permit independent rotation of the third section relative to the fourth section, the second swivel interface including the second inner sealing surface of the third section and the second outer sealing surface of the fourth section,
the second and fourth sections form opposite ends of an upper portion of the tee fitting.

8. The connector of claim 7, wherein the tee fitting comprises a lower portion substantially perpendicular to and fluidly connected to the upper portion.

9. The connector of claim 1, wherein the outer sealing surface is configured to frictionally engage the inner sealing surface.

10. The connector of claim 1, wherein the outer sealing surface has a width that is at least 15% of an inner diameter of the first section.

11. The connector of claim 1, wherein the inner sealing surface has a width that is greater than the width of the outer sealing surface.

12. The connector of claim 1, wherein the second section comprises a male coupling end including the outer coupling surface and at least one tab configured to engage with a shoulder formed in the female coupling end when the first and second sections are connected.

13. The connector of claim 1, wherein the first male coupling surface is located outboard of the second male coupling surface.

14. The connector of claim 1, wherein the swivel interface is configured such that a stream of gas passing through the connector at a pressure of between 10 to 15 psia experiences a pressure drop due to loss of the stream of gas through the swivel interface of less than 1 psia.

15. The connector of claim 1, wherein the first and second male coupling surfaces are connected by an outwardly projecting shoulder.

16. The connector of claim 1, wherein each of the first and second male coupling surfaces are of constant outer diameter across the widths thereof, and across the entirety of the widths thereof.

17. The connector of claim 1, wherein the connector is a straight connector.

18. An assembly comprising:
a connector according to claim 1;
a first piece of respiratory equipment attached to the first section; and
a second piece of respiratory equipment attached to the second section.

19. The assembly of 18, wherein at least one of the first and second pieces of respiratory equipment is configured to be connected to a nebulizer.

20. The assembly of claim 18, wherein the first piece of respiratory equipment comprises a hose or tubing, and the second piece of respiratory equipment comprises at least a portion of a tee fitting.

21. A method of joining two pieces of respiratory equipment together comprising:
providing a connector comprising:
a first section comprising first and second coupling ends, the first coupling end comprising a female coupling end with an inner sealing surface and the second coupling end comprising first and second male coupling surfaces, the first male coupling surface having a smaller outer diameter than the second male coupling surface,
a second section comprising an outer sealing surface, the first and second sections being connected by a swivel interface including the inner sealing surface of the first section and the outer sealing surface of the second section to permit independent rotation of the first section relative to the second section;
attaching a female end of a first piece of respiratory equipment to the first section; and
fluidly connecting the first section to a second piece of respiratory equipment via the second section.

22. The method of claim 21, wherein the first piece of respiratory equipment comprises a hose or tubing, and wherein the second piece of respiratory equipment comprises at least a portion of a tee fitting.

23. The method of claim 21, wherein at least one of the first and second pieces of respiratory equipment are configured to be connected to a nebulizer, and wherein the nebulizer is operated to deliver a medication to a patient, the medication traversing through the connector.
